(19) United States
(12) Patent Application Publication
(10) Pub. No.: US 2019/0111026 A1
(43) Pub. Date: Apr. 18, 2019

(54) ANTI-TRITRICHOMONAS AGENT

(71) Applicants: Pola Pharma Inc., Tokyo (JP); Nihon Nohyaku Co., Ltd., Tokyo (JP)

(72) Inventors: Yoshiyuki MIYATA, Tokyo (JP); Takaaki MASUDA, Yokohama-shi, Kanagawa (JP)

(21) Appl. No.: 16/090,129

(22) PCT Filed: Oct. 12, 2016
(86) PCT No.: PCT/JP2016/004550

§ 371 Date: Sep. 28, 2018

(30) Foreign Application Priority Data
Mar. 31, 2016 (JP) 2016-070255

(51) Int. Cl. A61K 31/4178; A61P 33/02

(52) U.S. Cl. CPC A61K 31/4178; A61K 9/14; A61P 33/02

(57) ABSTRACT

An object of the present invention is to provide a novel anti-Tritrichomonas agent. Provided is an anti-Tritrichomonas agent containing luliconazole.

### TECHNICAL FIELD

The present invention relates to an anti-Tritrichomonas agent and luliconazole as an active ingredient of the anti-Tritrichomonas agent.

### BACKGROUND ART

Protozoa of the genus Tritrichomonas (also referred to as Tritrichomonas protozoa hereinafter) are highly free-living protozoa which belong to flagellata. Protozoa of the genus Tritrichomonas include Tritrichomonas foetus, Tritrichomonas mobilensis and the like, and they are very similar in morphology. Tritrichomonas foetus is responsible for spontaneous abortion in cows and diarrhea in cats and pigs. In addition, although the causal relationship with diseases is not known very much, Tritrichomonas mobilensis is known to cause an ulcer in a gastrointestinal, tract due to coinfection, for example (see, for example, Non-Patent Documents 1 and 2). Furthermore, these protozoa of the genus Tritrichomonas are also known to cause opportunistic infections in humans. In the opportunistic infection, the infection site is mainly a gastrointestinal tract, and the infection begins with symptoms of diarrhea and develops into a systemic infection as it gets severe (see, for example, Non-Patent Document 3).

Among Trichomonas protozoa, Trichomonas vaginaris of the genus Tetratrichomonas is a cause of vaginitis. Usually a vaginal tablet or an injection of metronidazole is used as a therapeutic agent for Trichomonas vaginaris. However, metronidazole cannot be used for protozoa of the genus Tritrichomonas in the intestinal tract because metronidazole does not work against Trichomonas protozoa in the intestinal tract. In other words, the development of a drug that works effectively against protozoa of the genus Tritrichomonas in the intestinal tract has been desired (see, for example, Non-Patent Document 4). Specifically, a drug has been demanded which reaches stably the inside of the intestinal tract and works effectively against protozoa of the genus Tritrichomonas.

On the other hand, luliconazole is known to display antiprotozoal activity against highly parasitic protozoa of the genus Tetratrichomonas such as Trichomonas vaginaris (see, for example, Patent Document 1). However, no activity against highly free-living protozoa of the genus Tritrichomonas has been known.

### CITATION LIST

### Patent Literature

Patent Literature 1: JP 2016-504987 A

### Non Patent Literature

Non Patent Literature 1: Midlej V. et. al., Vet. Parasitol. 182: 171-180.

Non Patent Literature 2: Doi, J. et, al., 2012. J. Vet. Med. Sci. 74: 413-417.

Non Patent Literature 3: Suzuki J, et, al., J. Vet. Med. Sci. Article ID: 15-0644.

Non Patent Literature 4: Schroeder M S., Am Fam Physician. 71: 921-8 (2005).

### SUMMARY OF INVENTION

### Technical Problem

The present invention is provided under the condition as stated above and has an object of providing a drug that works effectively against Tritrichomonas protozoa.

### Solution to Problem

Considering this situation, the inventors have made a dedicated effort in search of a drug that works effectively against Tritrichomonas protozoa, especially a drug that reaches stably the inside of the intestinal tract and works effectively against Tritrichomonas protozoa. In consequence, they have found that a formulation which contains luliconazole as an active ingredient has such an effect and have completed the present invention. The present invention is described as follows.
[1] An anti-Tritrichomonas agent containing luliconazole.
[2] The anti-Tritrichomonas agent according to [1], wherein Tritrichomonas protozoa are Tritrichomonas foetus or Tritrichomonas mobilensis.
[3] The anti-Tritrichomonas agent according to [1] or [2], wherein the agent is for intestinal trichomoniasis.
[4] The anti-Tritrichomonas agent according to any one of [1] to [3], wherein the luliconazole is present in the agent in the form of a solid.
[5] The anti-Tritrichomonas agent according to [4], wherein the luliconazole present in the agent has an average particle size of not more than 100 μm.
[6] The anti-Tritrichomonas agent according to [5], wherein the luliconazole present in the agent has an average particle size of from 70 μm to 100 μm.
[7] The anti-Tritrichomonas agent according to any one of [1] to [6], wherein the agent is for human or an animal.
[8] Luliconazole having an average particle size of not more than 100 μm.
[9] The luliconazole according to [8], wherein the average particle size is from 70 μm to 100 μm.
[10] The luliconazole having, in the form of a solid, a growth inhibition rate of not less than 80% against Tritrichomonas protozoa.
[11] The luliconazole according to any one of [8] to [10], which is for use as an anti-Tritrichomonas agent.
[12] The luliconazole according to [11], which is for use an anti-Tritrichomonas agent for human or an animal.
[13] A method for producing an active ingredient for a pharmaceutical comprising luliconazole as the active ingredient, comprising:

recrystallizing luliconazole from a recrystallizing solvent comprising alcohols; and

measuring an average particle size of luliconazole crystal, and selecting as the active ingredient when the average particle size of luliconazole crystal is not more than 100 μm.

### Advantageous Effects of Invention

According to the present invention, a novel drug can be provided which works effectively against Tritrichomonas protozoa. Moreover, according to the present invention, a drug can be provided which reaches stably the inside of an intestinal tract and works effectively against Tritrichomonas protozoa.

### DESCRIPTION OF EMBODIMENTS

### <1> Anti-Tritrichomonas Agent

The anti-Tritrichomonas agent of the present invention is characterized in that the agent contains luliconazole. Luliconazole has a structure as shown below, and has already been available on the market and utilized as an anti-fungal agent.

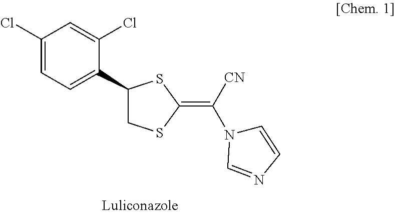 [SMILES: N#C/C(=C1/SC[C@@H](c2ccc(Cl)cc2Cl)S1)n1ccnc1]

Luliconazole as shown above can be synthesized according to a method disclosed in, for example, JP 60-218387 A. Particularly luliconazole can be synthesized in a method described in the following formula. More specifically, a compound represented by the general formula (1) can be provided by reacting 1-cyanomethyl imidazole and carbon disulfide to obtain a compound (III), and then reacting the compound (III) with a compound of the general formula (II) having a leaving group. After the reaction, the resultant may be purified according to a conventional recrystallization method.

In the following formula, R and X are a hydrogen atom or a halogen atom. Among compounds represented by this general formula (1), the compound in which R═X═Cl is luliconazole. Examples of leaving groups Y and Y′ suitably include, for example, methanesulfonyloxy group, benzensulfonyloxy group, p-toluenesulfonyloxy group and a halogen atom (see the following scheme).

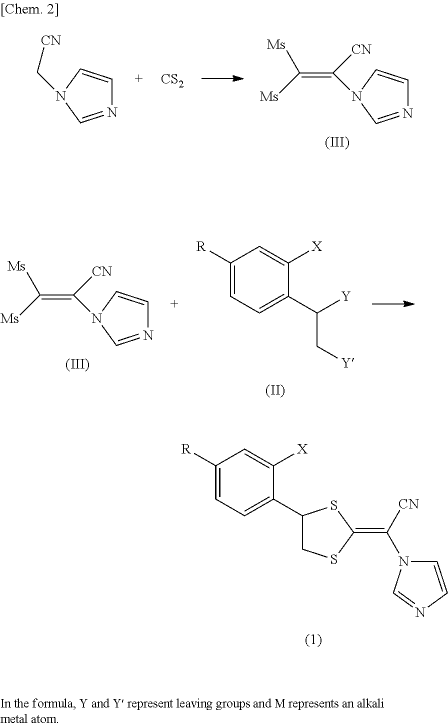

Luliconazole has excellent antiprotozoal activity against Tritrichomonas protozoa. Since luliconazole is insoluble in water, it is believed that luliconazole reaches the intestinal tract stably when administered orally (see Lulicon (registered trademark) Cream Pharmaceutical Product Interview Form 2006, revised edition, page 4). Furthermore, since luliconazole exhibits excellent antiprotozoal activity against Tritrichomonas protozoa not only in the form of a liquid but also in the form of a solid, when processed as a solid formulation and orally administered, luliconazole can reach the intestinal tract in an intact form and inhibit the growth of Tritrichomonas protozoa in the intestinal tract.

The form of the solid luliconazole is, in terms of its anti-Tritrichomonas effect, preferably in the form of powder having an average particle size of not more than 100 μm, more preferably in the form of powder having an average particle size of 70 μm to 100 μm, and even more preferably in the form of powder having an average particle size of 75 μm to 100 μm. The powder may be crystalline or amorphous.

The powder as described above can be obtained by for example, grinding a luliconazole crystal. Examples of the grinding methods include, for example, a grinding machine equipped with an agate mortar, grinding with a medium mill such as a planetary ball mill or a dyno mill, and grinding by a jet mill. The resulting average particle size varies depending on the bulk to be used even under the same grinding conditions, and strict grinding conditions do not necessarily provide a fine average particle size. Thus, the average particle size cannot be replaced by grinding conditions. Therefore, it is desired that the average particle size is actually measured and then it is determined whether or not luliconazole having such average particle size is applied to an anti-Tritrichomonas agent. The average particle size of luliconazole can be obtained by measuring the number average particle size. The number average particle size can be measured as an average size of particles through analysis of an image from a microscope.

For example, the average particle size is measured according to the following procedure. First of all, as an inverted microscope, Diaphot inverted microscope (manufactured by Nikon Corporation) is used and the powder of luliconazole is observed. Some particles are then selected arbitrarily and measured for particle size. It is noted that 100 particles or more are measured.

Moreover, the average particle size of luliconazole can be also obtained as an average particle size of the accumulated volume as measured and obtained by a laser diffraction particle size distribution analyzer.

By employing such a mode as above, luliconazole which is an active ingredient of the present invention exhibits, in the form of a solid, not less than 80% of the growth inhibition rate against Tritrichomonas protozoa, in particular Tritrichomonas mobilensis. In other aspects, luliconazole of the present invention is luliconazole which shows the growth inhibition rate of not less than 80%, more preferably not less than 85%, even more preferably not less than 88% is. It is noted that the growth inhibition rate is calculated as follows: [(the number of viable cells of the control group−the number of viable cells of the sample group)/the number of viable cells of the control group]×100. It is preferred that the anti-Tritrichomonas effect is determined 24 to 72 hours after the inoculation of 10,000 cells of Tritrichomonas protozoa on 5 mL of liquid medium in which 5 mg of sample luliconazole is added. As the liquid medium, it is preferred to use Diamond's medium with 5% to 15% adult bovine serum added as desired.

<2> Formulation of an anti-Tritrichomonas Agent

The anti-Tritrichomonas agent of the present invention can be processed into a variety of formulations and can be of practical use. Suitable forms of the formulations include dosage forms in which luliconazole powder having an average particle size of not more than 100 μm, more preferably an average particle size 70 μm to 100 μm, even more preferably an average particle size 75 μm to 100 μm, is provided in the intestinal tract. Suitable examples of such dosage forms include, for example, solid formulations such as tablets and granules obtained by processing powder having an average particle size of not more than 100 μm, more preferably powder having an average particle size 70 μm to 100 μm, or even more preferably powder having an average particle size 75 μm to 100 μm along with an excipient or a disintegrant. In addition, the suitable examples also include solid formulations such as capsules obtained by filling luliconazole powder in a capsule.

Examples of the excipient suitably include, for example, lactose and croscarmellose; examples of the disintegrant suitably include, for example, starch and macrocrystalline cellulose. A binding agent such as hydroxypropylcellulose (HPC) and hydroxypropylmethylcellulose (HPMC) or a lubricant such as magnesium stearate can be appropriately added. The content of the anti-Tritrichomonas agent in a formulation is preferably 10% to 90% by mass, and more preferably 20% to 80% by mass. These ingredients can be treated according to a conventional method and processed into a formulation such as a granule, tablet or capsule, The agent can be also coated with a coating agent as appropriate.

The anti-Tritrichomonas agent of the present invention is preferably administered orally because the agent targets Tritrichomonas protozoa in the intestinal tract. The dosage by oral administration varies depending on the symptoms and the like, but anti-Tritrichomonas agent of the present invention is preferably administered in an amount of approximately 100 mg to 10,000 mg per adult, at one time or several times dividedly. Thus, the oral administration of the formulation of the present invention allows the anti-Tritrichomonas agent of the present invention to reach the infection site in the intestinal tract and to exterminate the cause of diarrhea, or Tritrichomonas protozoa.

### EXAMPLES

The present invention will be further explained in detail below by way of examples; however, the present invention is not limited to these examples as long as they do not deviate the gist of the present, invention.

### Example 1

The activity of luliconazole against Tritrichomonas mobilensis of the genus Tritrichomonas (T. mobilensis: USA: M776 c12) was studied. Luliconazole as synthesized according to the method disclosed in the above JP 60-218387 A was used as a sample. More specifically, Tritrichomonas protozoa were cultured on Diamond's medium with 10% adult bovine serum added. Luliconazole was dissolved in 100 μL of methanol to obtain solutions having a variety concentrations of luliconazole. The cultures were then inoculated on 5 mL of 10% adult bovine serum-added Diamond's media in which the solutions were added, cultured at 35° C. for 24 hours, and then measured for the number of viable Tritrichomonas protozoa by use of a Neubauer hemocytometer. MIC and IC₅₀ were obtained from the number of viable protozoa. MIC was 50 μg/mL and IC₅₀ was 10. 1 μg/mL. This result indicates that luliconazole exhibits an excellent antiprotozoal effect in a state of solution.

### Example 2

Luliconazole was ground with a mortar to change the particle size, and the anti-Tritrichomonas activity of solid luliconazole was studied. More specifically, luliconazole having a variety of particle sizes was added in an amount of 5 mg to 5 mL of Diamond's media supplemented with 10% adult bovine serum, and 10,000 cells of Tritrichomonas mobilensis were inoculated on the media, cultured at 35° C. for 24 hours, and then measured for the number of viable T. mobilensis cells by use of a Neubauer hemocytometer. Luliconazole was not added to the control group. The growth inhibition rate was calculated as follows: [(the number of viable cells of the control group−the number of viable cells of the sample group)/the number of viable cells of the control group]×100. The results are shown in Table 1. The results indicate that, even in the form of a solid, luliconazole exhibits antiprotozoal activity and that a preferable particle size is not more than 100 μm. It is noted that the particle size was measured by a laser diffraction method (Microtrac MT3000, manufactured by NIKKISO CO., LTD; dispersion medium: water).

These results show that luliconazole exhibits, even in the form of a solid, excellent anti-protozoal effect on Tritrichomonas protozoa. Therefore, it is believed that luliconazole reaches stably the inside of the intestinal tract and thus, luliconazole displays effectively anti-Tritrichomonas effect even in the intestinal tract.

TABLE 1

| | | Viable | | Growth |
|---|---|---|---|---|
| | | Cell Count | | Inhibition |
| Sample | D₅₀ (μm) | Ave. | SD | Rate |
| Control | — | 176,667 | 5,774 | — |
| 5-minute ground | 114.4 | 43,333 | 5,774 | 75.5 |
| luliconazole | | | | |
| 6-minute ground | 96.6 | 20,000 | 10,000 | 88.7 |
| luliconazole | | | | |
| 7-minute ground | 84.7 | 20,000 | 17,321 | 88.7 |
| luliconazole | | | | |
| 8-minute ground | 78.5 | 26,667 | 11,547 | 84.9 |
| luliconazole | | | | |

### Example 3

In order to confirm the influence of luliconazole which was eluted in the solutions in Example 2, 7 minute-ground luliconazole was added in an amount of 5 mg to Diamond's medium with 10% adult bovine serum added, and stored at 35° C. for 24 hours, and the supernatant was obtained from the Diamond's medium. Tritrichomonas mobilensis was inoculated on the supernatant, cultured at 35° C. for 24 hours and measured for the number of viable cells by use of a Neubauer hemocytometer. Luliconazole was not added to the control group. The result from the control group was 973,333±23,094 while the result from the supernatant was 1,000,000±144,222. It was obvious that the supernatant did not have anti-Tritrichomonas effect and that luliconazole worked in the form of a solid. In this regard as well, it is clear that luliconazole can control effectively Tritrichomonas protozoa in the intestinal tract.

### Example 4

A variety of ground luliconazole from Example 2 was used to prepare formulations. Specifically a variety of ground luliconazole (5 minute-ground luliconazole, 6 minute-ground luliconazole, 7 minute-ground luliconazole, and 8 minute-ground luliconazole) was individually filled in a capsule of 100 mg and example formulations 1 to 4 were prepared.

### INDUSTRIAL APPLICABILITY

The present invention can be applied in a pharmaceutical, veterinary drug and the like.

1. (canceled)

2. (canceled)

3. (canceled)

4. (canceled)

5. (canceled)

6. (canceled)

7. (canceled)

8. Luliconazole having an average particle size of not more than 100 μm.

9. The luliconazole according to claim 8, wherein the average particle size is from 70 μm to 100 μm.

10. Luliconazole having, in the form of a solid, a growth inhibition rate of not less than 80% against Tritrichomonas protozoa.

11. Luliconazole according claim 8, which is for use as an anti-Tritrichomonas agent.

12. Luliconazole according to claim 11, which is for use as an anti-Tritrichomonas agent for human or an animal.
